(19) United States
(12) Patent Application Publication
(10) Pub. No.: US 2012/0271121 A1
(43) Pub. Date: Oct. 25, 2012

(54) Integrated Biometric Sensing and Display Device

(75) Inventors: Marco Kenneth Della Torre, San Francisco, CA (US); Matthew Wayne Eckerle, Oakland, CA (US); Jean Louise Rintoul, San Francisco, CA (US); Claus He, Shenzhen (CN); Bashir Ziady, San Mateo, CA (US); Andrew Atkinson Stirn, San Francisco, CA (US); Nadeem Iqbal Kassam, San Francisco, CA (US); Steven Paul Harris, San Francisco, CA (US); Sean Tan, San Jose, CA (US); Christopher James Verplaetse, San Francisco, CA (US)

(73) Assignee: BASIS Science, Inc., San Francisco, CA (US)

(21) Appl. No.: 13/336,233

(22) Filed: Dec. 23, 2011

Related U.S. Application Data
(60) Provisional application No. 61/428,036, filed on Dec. 29, 2010.

(51) Int. Cl. A61B 5/0205; A61B 5/05; A61B 5/11; A61B 5/024; A61B 5/026; A61B 5/1455; A61B 5/021; A61B 5/01; A61B 6/00

(52) U.S. Cl. 600/301; 600/476; 600/479

(57) ABSTRACT

A biometric device configured to be attached to a portion of a body of a user measures biometric data of the user. The device includes an optical emitter, a wavelength filter, an optical sensor and a processor, for sending a light to the body of a user, receiving light received from the user, filtering and processing it to measure biometric data of the user, including for example, heart rate and blood flow rate. In addition, the biometric device may include other sensors, such as a galvanic skin response sensor, an ambient temperature sensor, skin temperature, motion sensor, etc., to enable the biometric device to measure arousal or conductivity changing events, ambient temperature, user temperature and motion associated with the user. Additionally, information from each sensor may be used to further filter noise in one or more signals received by the sensors to provide biometric data to the user.

### CROSS REFERENCE TO RELATED APPLICATIONS

This application is related to U.S. Patent Application No. 61/428,036, filed Dec. 29, 2010, and titled “Integrated Biometric Sensing and Display Device,” the contents of which are hereby incorporated by reference.

### BACKGROUND

1. Field of Art

The disclosure generally relates to the field of signal processing and more specifically to measuring biometric data of a person at a location away from the heart.

2. Description of the Related Art

Cardiovascular parameters, such as heart rate may be measured by electrocardiographic sensing devices or by pressure sensing devices, among others. Optical sensing devices, for example, transmit a light to the person's body tissues and employ an optical sensor to measure the light transmitted through, or received back, from the body tissues. Due to the pulsing of the blood flow or other body fluids, the devices can typically calculate the person's pulse rate based on a measure of the light sensed back from body tissues. Advantages of these devices are that they are non-invasive and they can monitor the relevant parameters on a continuous basis. However, such devices are typically ineffective at managing the effects of noise sources that mask the signal to be monitored. The most common such noise sources include the motion of the wearer and ambient light interference. This results in poor measurement accuracy and, therefore strongly limits the utility of such devices.

Electrocardiographic sensing devices measure electrical impulses to detect cardiovascular parameters of an individual. However, such devices typically see spurious noise in measuring electrical impulses from an individual. One solution to the spurious noise is to place the electrocardiographic device near a person's heart where signal to noise ratio is the highest. However, such a placement generally requires a chest-strap device which is often cumbersome for the user. For example, such devices are inconvenient to wear during everyday life and thus are typically used only during limited periods of activity. Therefore, such devices often do not capture a user's biometric data during vast majority of the day. As such, electrocardiographic sensing systems typically do not provide a complete picture of a person's biometric data throughout the day. A more continuous, complete picture of a person's biometric data has much greater value, as it captures the body's response to all aspects of life, rather than limited periods alone.

Some electrocardiographic sensing devices offer a single unit solution wherein a person's heart rate is monitored and displayed at the person's wrist when the user touches or activates a sensor on the sensing device. As such, the devices also do not provide continuous measurement of a user's heart rate. Furthermore, such measurement often requires the user's active involvement in the measurement process, rather than being continuous and passive.

### BRIEF DESCRIPTION OF DRAWINGS

The disclosed embodiments have other advantages and features, which will be more readily apparent from the detailed description, the appended claims, and the accompanying figures (or drawings). A brief introduction of the figures is below.

FIG. 1 illustrates one embodiment of a device to capture biometric data from a user.

FIG. 2 illustrates one embodiment of components of an example machine able to read instructions from a machine-readable medium and execute them in a processor (or controller).

FIG. 3 illustrates a block diagram of an optical sensor for receiving optical signals, in accordance with one embodiment.

FIG. 4 illustrates a block diagram of a processor enabled to receive biometric data from sensors to optimize an input signal, in accordance with one embodiment.

FIG. 5 illustrates a process for measuring a biometric data of a user based on data measured by one or more sensors.

FIG. 6 illustrates an example embodiment of a device housing sensors to capture biometric data from a user.

### DETAILED DESCRIPTION

The Figures (FIGS.) and the following description relate to preferred embodiments by way of illustration only. It should be noted that from the following discussion, alternative embodiments of the structures and methods disclosed herein will be readily recognized as viable alternatives that may be employed without departing from the principles of what is claimed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

### Configuration Overview

One embodiment of a disclosed system, method and computer readable storage medium that includes measuring biometric data of a user using a device attached to a portion of a body of a user, for example, an appendage (or limb). The system, method and computer readable storage medium include transmitting light to skin of a user, receiving light received from body tissues and bodily fluids of a user, filtering the light and sensing the filtered light to measure biometric data of the user. By combining optical signals with signals from other sensors, the device is enabled to identify the light being reflected or received from flowing blood and filter signal noise caused by ambient light, user movement, etc. In one embodiment, the sensor used to measure signal noise source is a motion sensor such as an accelerometer, such that the optical signal can be separated into a component relating to motion-induced noise and another component relating to blood flow. As described in greater detail in the specification, algorithmic techniques may also be used to filter out the noise, such as dynamic tracking of rates to guide intelligent peak detection algorithms.

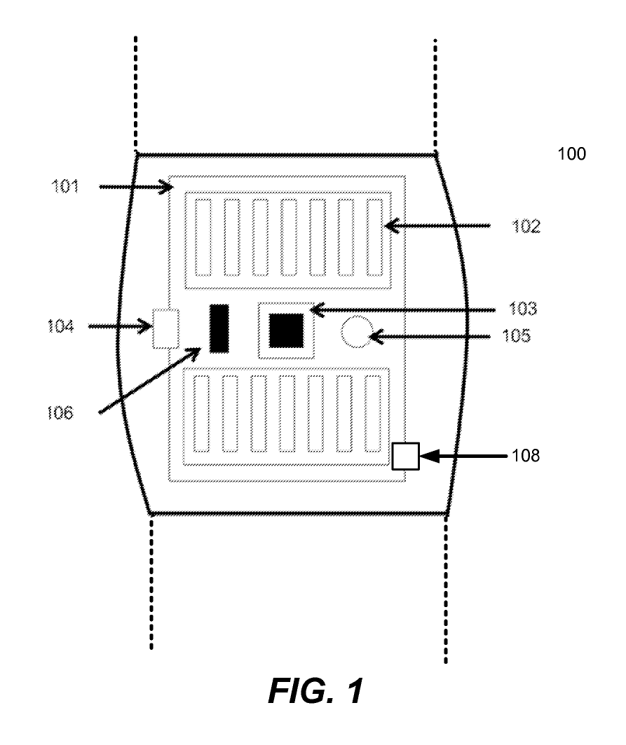

FIG. 1 illustrates one embodiment of a device 100 to capture biometric data from a user. The device includes a galvanic skin response (GSR) sensor 102, an optical sensor 103, an ambient temperature sensor 104, motion sensor 105, a skin temperature sensor 106, an energy harvesting module 108 and bands 110 for securing the device to a body of a user. The sensors are placed (or housed) within a sensor housing component 101. In one embodiment, the housing component 101 is configured to couple to a user, e.g., through a wristband or armband, so that the sensors are exposed to collect information in the form of data from the users. The sensors are used to capture various types of information and produce output signals which may be analyzed to calculate various biometric data about the user. In addition, information from one or more sensors may be used to further filter noise at other sensors. As such, the sensors collectively improve the accuracy of the sensors within the device 100.

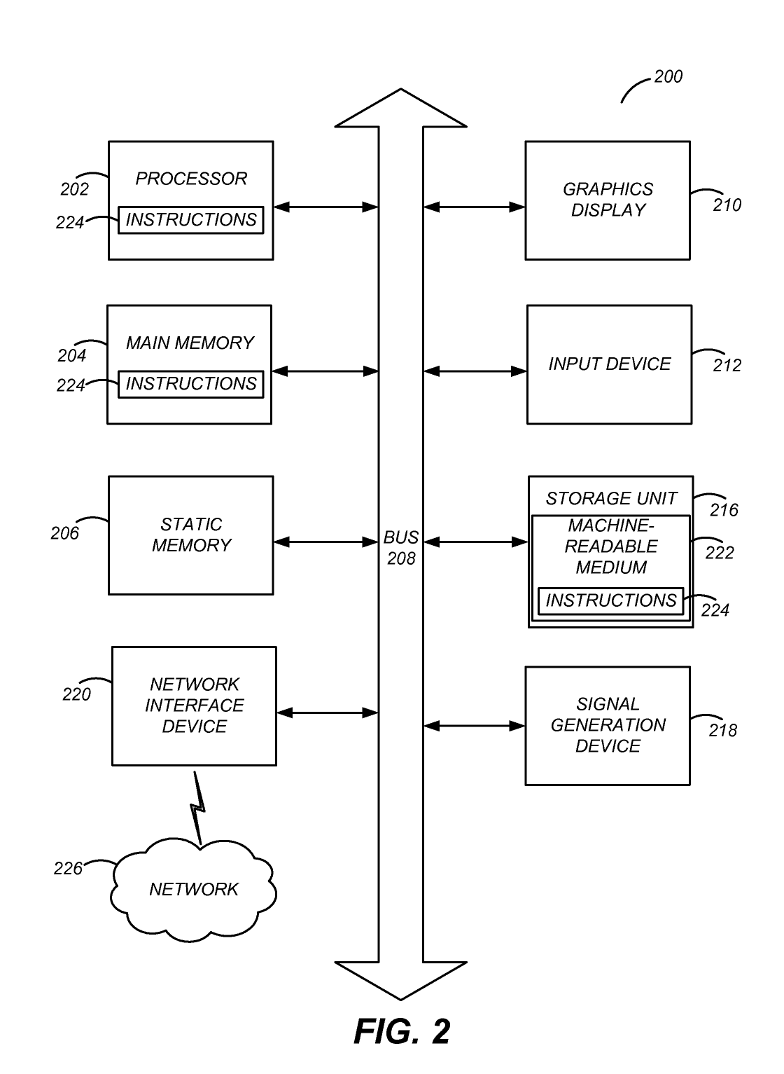

As noted, the sensors detect (or collect) information corresponding to their particular function. The information collected from the sensors is provided to a processor, which uses the data to derive various biometric data about a user. The processor is described in greater detail in reference to FIG. 2. In other embodiments, a different type, number, orientation and configuration of sensors may be provided within the housing component 101.

Referring now to the sensors in more detail, the GSR sensor 102 detects a state of a user by measuring electrical conductance of skin, which varies with its moisture or sweat levels. A state of a user may be characterized by changes associated with physical activity, emotional arousal or other conductivity changing events. For example, since sweat glands are controlled by a sympathetic nervous system, sweat or electrical conductance may be used as an indication of a change in the state of a user. Thus, in one instance, the GSR sensor 102 measures galvanic skin response or electrical conductance of skin of a user to identify a change in the state of a user. In one embodiment, the GSR sensor 102 passes a current through the body tissue of a user and measures a response of the body tissue to the current. The GSR sensor 102 can calculate skin conductivity of a user based on the measured response to the electric current. The GSR sensor 102 may also measure a sweat levels of a user. The sweat levels, along with other user provided information may be used to determine caloric burn rates of a user and characterize exercise parameters. In other embodiments, the GSR sensor 102 identifies a change in a state of the user based on detected sweat levels as well as input signals received from other sensors included in the housing component 101. For example, a sharp change in ambient temperature detected by the ambient temperature sensor 104 may indicate that a sharp increase in sweat levels of a user may not be caused by a change in the state of a user but rather because of a change in the ambient temperature. In one embodiment, the GSR sensor 102 sends the calculated conductivity information to a processor as an electrical signal.

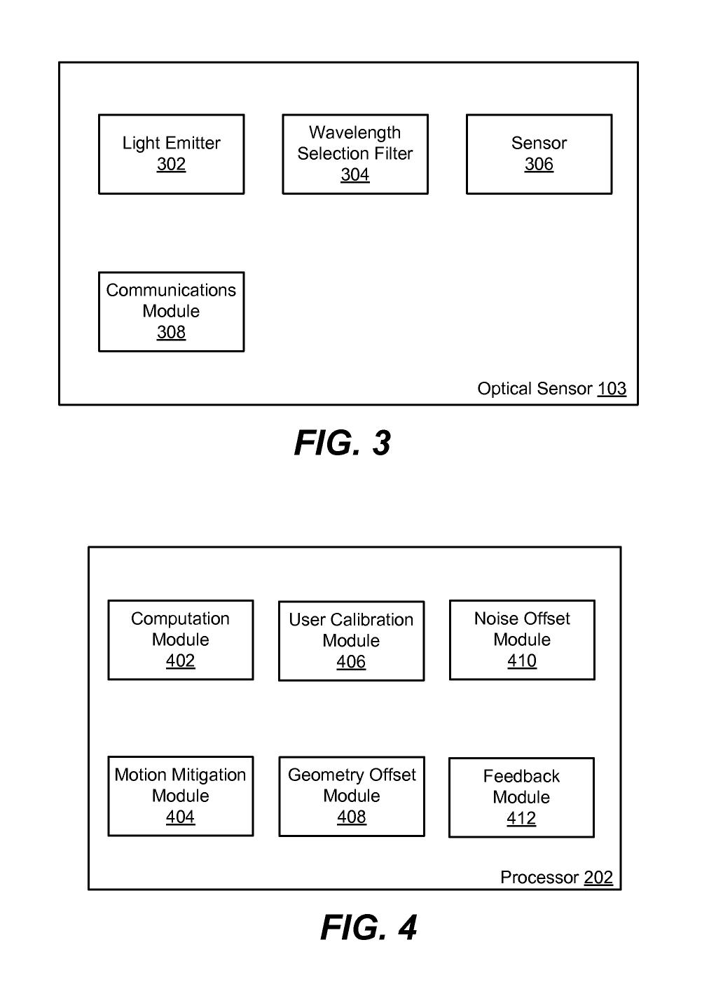

The optical sensor 103 measures heart rate of a user by measuring a rate of blood flow. In one embodiment, the optical sensor 103 sends a signal to skin and tissue of the user and receives the reflected light from the body of the user to measure a blood flow rate. In one embodiment, the sensor converts the light intensity into voltage. The light intensity as reflected from the body of the user, varies as blood pulses under the sensor, since the absorbance of light, including for example, green light is altered when there is more blood underneath the sensor as opposed to less. This voltage is converted to a digital signal which may be analyzed by a processor for regular variations that indicates the heart's pulsation of blood through the cardiovascular system. Additionally, the blood flow rate captured by the optical sensor 103 may be used to measure other biometric data about the user, including but not limited to beat-to-beat variance, respiration, beat-to-beat magnitude and beat-to-beat coherence. The optical sensor 103 is described in greater detail in reference to FIG. 3.

The ambient temperature sensor 104 detects temperature surrounding the user or the biometric device and converts it to a signal, which can be read by another device or component. In one embodiment, the ambient temperature sensor 104 detects the temperature or a change in temperature of the environment surrounding the user. The ambient temperature sensor 104 may detect the temperature periodically, at a predetermined frequency or responsive to instructions provided by a processor. For example, a processor may instruct the ambient temperature sensor 104 to detect temperature when activity is detected by a motion sensor 105. Similarly, the ambient temperature sensor 104 may report the detected temperature to another device at a periodic interval or when a change in temperature is detected. In one embodiment, the temperature sensor 104 provides the temperature information to a processor. In one embodiment, the ambient temperature sensor 104 is oriented in a manner to avoid direct contact with a user when the user wears the device 100.

The motion sensor 105 detects motion by measuring one or more of rectilinear and rotational acceleration, motion or position of the biometric device. In other embodiments, the motion sensor may also measure a change in rectilinear and rotational speed or vector of the biometric device. In one embodiment, the motion sensor 105 detects motion along at least three degrees of freedom. In other embodiments, the motion sensor 105 detects motions along six degrees of freedom, etc. The motion sensor 105 may include a single, multiple or combination axis accelerometer to measure the magnitude and direction of acceleration of a motion. The motion sensor 105 may also include a multi-axis gyroscope that provides orientation information. The multi-axis gyroscope measures rotational rate (d(angle)/dt, [deg/sec]), which may be used to determine if a portion of a body of the user is oriented in a particular direction and/or be used to supplement information from an accelerometer to determine a type of motion performed by the user based on the rotational motion of a user. For example, a walking motion may cause a ‘pendulum’ motion at a wrist of the user, whereas a running motion may cause a cyclic motion at the user wrist along an axis lateral to a direction detected by an accelerometer. Additionally, the motion sensor 105 may use other technologies such as magnetic fields to capture orientation or motion of a user along several degrees of freedom. In one embodiment, the motion sensor 105 sends electrical signals to a processor providing direction and motion data measured by the sensor 105. In one embodiment, the motion detected by the motion sensor 105 is used to filter signal noise received by the optical sensor 103. For example, motion detected at a particular time may be used to discount a peak signal detected by an optical sensor at the same time because the peak signal detected by the optical sensor 103 is likely related to the motion of the user and not the heart beat of the user.

The skin temperature sensor 106 measures skin temperature of a user. In one embodiment, the biometric device and the skin temperature sensor 106 come in contact with skin of a user, wherein the skin temperature sensor 106 takes a reading of skin temperature of the user. In one embodiment, the skin temperature sensor 106 detects the temperature or a change in skin temperature of the user. The skin temperature sensor 106 may detect the temperature periodically, at a predetermined frequency or responsive to instructions provided by a processor. For example, a processor may instruct the skin temperature sensor 106 to detect temperature when activity is detected by the motion sensor 105. Similarly, the skin temperature sensor 106 may report the detected temperature to another device at a periodic interval or when a change in temperature is detected. In one embodiment, the temperature sensor 104 provides the temperature information to a processor.

The energy harvesting module 108 converts energy received from the environment surrounding the device 100 to electrical energy to power the device 100. In one embodiment, the power harvested by the energy harvesting module 108 may be stored in one or more batteries housed on the device 100. The energy harvesting module 108 may convert electrical energy from a variety of sources, including, but not limited to mechanical energy from movements generated by a user, static electrical energy, thermal energy generated by the body of a user, solar energy and radio frequency (RF) energy from sources such amplitude modulated (AM), frequency modulated (FM), WiFi or Cellular Network signals. In one embodiment, the energy harvesting module 108 receives electrical energy from a power source with varying interfaces, such as a Universal Service Bus (USB) port or other proprietary interfaces. The energy harvesting module 108 may direct the energy to charge a battery housed on the device 100.

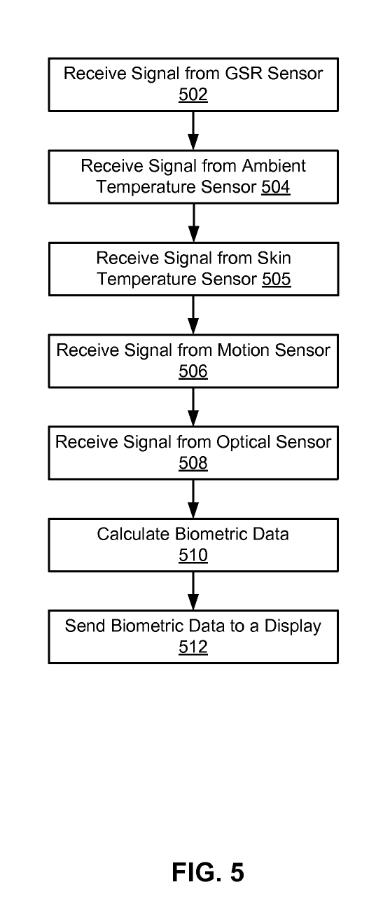

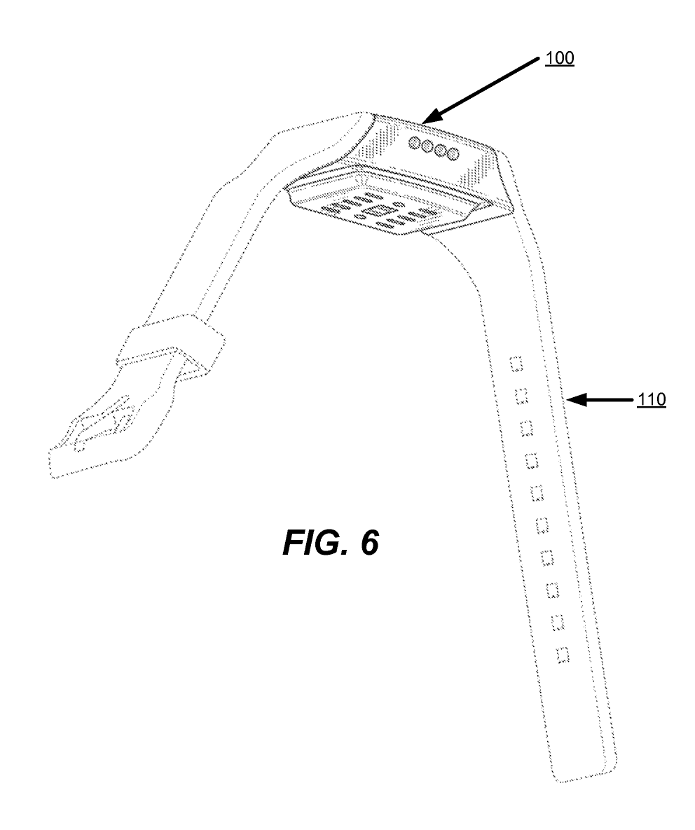

In one embodiment, the device 100 can be optionally attached to straps 110 for securing the device 100 to the body of a user. For example, the straps 110 can be used to secure the device 100 around a wrist, arm, waist, leg, etc., of a user. An exemplary embodiment of a device 100 with straps 110 is provided in reference to FIG. 6. Referring now to FIG. 6, the illustrated device 100 is an exemplary design used to house sensors that interface with a body of a user, such as the GSR sensor 102, the optical sensor 103, and skin temperature 106, as well as sensors that do not interface with the user such as the ambient temperature sensor 104, the motion sensor 105, and the energy harvesting module 108 as well as computing components described in reference to FIG. 2. It is noted that the embodiment illustrated in FIG. 5 is exemplary and the designs to house the sensors and the computing components in a device 100 may be implemented such that sensors interface with a body of a user and such that the device 100 attaches to straps 110 to secure the device to a body of a user.

### Computing Machine Architecture

As described with FIG. 1, the sensors detect (or collect) information that corresponds to data for processing by a processor housed in the device 100. FIG. 2 is a block diagram illustrating components of an example machine able to read instructions from a machine-readable medium and execute them in a processor (or controller). Specifically, FIG. 2 shows a diagrammatic representation of a machine in the example form of a computer system 200 encapsulated within the device 100, with instructions 224 (e.g., software) for causing the computer system 200 to perform any one or more of the methodologies discussed herein to be executed. Further, while only a single machine or computer device 200 is illustrated, the term “machine” or “computer device” shall also be taken to include any collection of machines that individually or jointly execute instructions 224 to perform any one or more of the methodologies discussed herein. The example computer system 200 includes a processor 202 (e.g., a central processing unit (CPU), a graphics processing unit (GPU), a digital signal processor (DSP), one or more application specific integrated circuits (ASICs), one or more radio-frequency integrated circuits (RFICs), one or more field programmable gate arrays (FPGAs) or any combination of these), a main memory 204, and a static memory 206, which are configured to communicate with each other via a bus 208. The computer system 200 may further include graphics display unit 210 (e.g., a plasma display panel (PDP), a liquid crystal display (LCD), a projector, or an organic light emitting diode (OLED) for displaying the data on the device 100 or on an external graphics display. The computer system 200 may also include an input device 212. The input device may include a touch screen, a keyboard, a trackball, or other sensors to enable a user to provide inputs to the device. In one embodiment, the device includes capacitive touch-pins on a surface to receive user inputs. In other instances, the input devices 212 include a GSR sensor 102, an optical sensor 103, an ambient temperature sensor 104, motion sensor 105 and a skin temperature sensor 106 configured to provide input signals to the computing device 200.

The computer system 200 also includes a storage unit 216, a signal generation device 218 (e.g., a speaker, vibration generator, etc.), and a network interface device 220, which also are configured to communicate via the bus 208. The storage unit 216 includes a machine-readable medium 222 on which is stored instructions 224 (e.g., software) embodying any one or more of the methodologies or functions described herein. The instructions 224 (e.g., software) may also reside, completely or at least partially, within the main memory 204 or within the processor 202 (e.g., within a cache memory of a processor) during execution thereof by the computer system 200, the main memory 204 and the processor 202 also constituting machine-readable media. The instructions 224 (e.g., software) may be transmitted or received over a network 226 via the network interface device 220.

In one embodiment, the network interface device 220 wirelessly connects to a network 226 and/or a computing device using any wireless networking technologies and protocols. The network interface device 220 may be a BLUETOOTH, WIFI, BTLE, ZIGBEE, Near Field Communications transceiver used to connect and exchange data with mobile computing devices. The network interface device 220 may provide connectivity directly to a network such as a cellular network using but limited to one or more of the GSM, CDMA, 3G and LTE protocols. Computing devices may include, for example, phones, smart phones, tablet computers, laptops, desktop computers, automotive systems, etc. In one embodiment, the network interface device 220 uploads data via a network 226 to a server that aggregates and displays the measured health information of a user in substantially real time. In another embodiment, the network interface device 220 receives contextual information which may include one or more of GPS, social and other data from computing devices wirelessly connected to the device 100, and saves this information on internal memory for display to the user and later transmission to a server. The server may aggregate the user data and the location based data to provided integrated information to a user on the device itself or via another device such as a smart-phone or internet site. For example, the server may provide that the average heart rate of a user is higher or lower when using a particular route to commute to work, by combining the heart rate measured by the device 100 and the location information sourced from another computing device. The server may also compile information from several users and provide an aggregated data of other users similarly situated to the user, either in substantially real time or at a later time and either on the device itself or on another computing device. Similarly, the network interface device 220 communicates with an automotive system that may display the recorded health data of a user on an automotive dashboard. The network interface device 220 may also interface with a mobile phone to initiate or augment a communication such as a Short Message Service (SMS) message, phone call, a posting of information to a social media application or to an emergency responder.

While machine-readable medium 222 is shown in an example embodiment to be a single medium, the term “machine-readable medium” should be taken to include a single medium or multiple media (e.g., a centralized or distributed database, or associated caches and servers) able to store instructions (e.g., instructions 224). The term “machine-readable medium” shall also be taken to include any medium that is capable of storing instructions (e.g., instructions 224) for execution by the machine and that cause the machine to perform any one or more of the methodologies disclosed herein. The term “machine-readable medium” includes, but should not be limited to, data repositories in the form of solid-state memories, optical media, and magnetic media.

### Sensing and Processing Configurations

FIG. 3 illustrates a block diagram of an optical sensor 103 for receiving optical signals, in accordance with one embodiment. The optical sensor 103 includes a light emitter 302, a wavelength selection filter 304, a sensor 306 and a communications module 308. In one embodiment, the optical sensor 103 measures light received from the body of a user, including tissues and bodily fluids, such as blood, and transmits the data to the processor 202 via a communications bus 208.

A light emitter 302 transmits a light source into the body tissue of a user. The light emitter 302 may include, but should not be limited to a light emitting diode (LED), a laser, an organic light emitting diode (OLED), electroluminescence sheet, etc. In one embodiment, the light emitter 302 may include more than one light emitter, wherein, each emitter may have the same or different emissions characteristics. The light produced by the light emitter 302 may be monochromatic, comprise multiple wavelengths on a broad spectrum, either visible, invisible or both. In one embodiment, the light emitter 302 emits lights onto the skin of a user. As further described in reference to FIG. 4 the light emitter 302 may output a signal responsive to instructions received from a processor. For example a processor 202 may provide instructions to change the output signal emitted by the light emitter 302 based on data provided by other sensors in the device 100. For example, if a sensor is unable to measure biometric data of a user because of excessive sunlight that may interfere with capturing light reflected from the user, the light emitter may be instructed to emit a different light frequency or emit light at a higher intensity. In one embodiment, the light produced by the light emitter 302 reflects against the body tissue of a user and is captured by the light sensor 306.

A wavelength selection filter 304 blocks frequencies of light allowing one or more isolated frequencies of light to pass to a sensor 306. In one embodiment, the wavelength selection filter 304 selects a wavelength for measuring blood flow optimally and provides the selected wavelength to the sensor 306. Similarly, the wavelength selection filter 304 may block visible or ultraviolet light and pass infrared light to the sensor 306. In one embodiment, the wavelength selection filter 304 may block all visible light but may permit mid-infrared wavelengths to pass. The wavelength selection filter 304 filters light emitted by the light emitter 302 and received from body tissue and body fluids of a user. As such, the wavelength selection filter 304 may be enabled to block sunlight, for example, to ensure that certain frequencies of light emitted by the light emitter 302 and received from the body tissues and body fluids of a user are captured for measuring the biometric data of a user. The particular frequencies filtered by the wavelength selection filter 304 may vary based on the frequencies of light emitted by the light emitter 302. The wavelength selection filter 304 may be implemented as a physical filter attached to the device 100. In such an instance, it may comprise a single or multi-filter array of passive filters, such as a thin-film filter, or one or more active optical filtering systems, each with similar or varying range of maximum and minimum reflectivity and transmission capabilities on two or more surfaces. In other embodiments, the wavelength selection filter 304 passes certain frequencies of light to enable the sensor to measure blood flow, blood oxygenation (SpO₂) and blood glucose levels of a user.

In one embodiment, the sensor 306 receives light that is received from body tissue of a user and passed by the wavelength selection filter 304. In one embodiment, the sensor 306 converts the received light to a pulse signal output, wherein the output is provided to a processor 202. In one embodiment, the communications module 308 interfaces with a communications bus 208 to send the pulse signal output to a processor. In one embodiment, light may be infrared (IR) light.

Turning now to FIG. 4, it illustrates a block diagram of one example embodiment of the processor 202 configured to receive biometric data from sensors to optimize an input signal. In this example embodiment, the processor 202 includes a computation module 402, motion mitigation module 404, a user calibration module 406, a geometry offset module 408, noise offset module 410 and a sensor feedback module 412. In one embodiment, the processor 202 receives signals from a galvanic skin response (GSR) sensor 102, an optical sensor 103, an ambient temperature sensor 104, a skin temperature sensor 106 and a motion sensor 105 to calculate biometric data associated with a user.

The computation module 402 receives information from each sensor housed in the device 100, including a GSR sensor 102, an optical sensor 103, an ambient temperature sensor 104, motion sensor 105, a skin temperature sensor 106 and compute biometric data to display to a user. For example, based on the blood flow rate measured by the optical sensor 103, the computation module 402 may compute heart rate, beat-to-beat variance, respiration rate, beat-to-beat magnitude and beat-to-beat coherence of a user. In one embodiment, based on a detection of heart beats from an measurement of blood flow, the processor computes a natural variance in beat to beat interval. The natural variance corresponds to a respiration rate of the user and is calculated by the computation module 402. In one embodiment, the computation module 402 computes a range over which heart beat intervals vary. The magnitude of the computed variance may be displayed to a user as a component in an assessment of one or more of the following: cardiovascular parameters, level of emotional arousal, occurrence of a stress event and level of stress event. In one embodiment, the computation module 402 analyses beat variance for regularity. For example, the computation module 402 determines whether the heart rate varies regularly between maximum and minimum interval beats or if the transition is erratic. In one embodiment, the computation module 402 measures a distance and speed of the user wearing the device 100 based on information provided by the motion sensor 105. For example, a distance may be detected by a combination of a step count and an estimate of stride length. Parameters such as stride length may also be provided by a user directly on the device or via another computing device, which transmits this information to be saved on the device via the network interface device 220. Additionally, the computation module 402 may also account for a detection of stairs, running, or other activities in determining distance travelled by a user. Similarly, a speed of the user may be determined by distance and time of travel for the user. The time factor may include, but is not limited to an activity period, a day, a week, etc.

The motion mitigation module 404 mitigates the impact of motion on the data captured by the optical sensor 103. In one embodiment, the motion mitigation module 404 receives data from the motion mitigation sensor 105 including information of the acceleration and direction of the motion of a user. For example, the motion mitigation module 404 may measure the extent and direction of tissue compression caused by motion of a user. In such an instance, the motion mitigation module 404 uses the tissue compression data to optimize the data captured by the optical sensor 103.

The user calibration module 406 receives one or more data streams about skin pigmentation, hair density and other parameters relevant to the user of the device, the environment around the device or user. This data is used to dynamically adjust sensor operation parameters or the way in which that data is processed, in order to optimize data captured by the sensors such as the optical sensor 103. For example, the skin pigmentation of a user may affect the data captured by the optical sensor 103. For example, light emitted by the light emitter 302 may reflect from the skin of a user at different intensities depending on the skin pigmentation of a user. As such, the pigmentation offset module 408 accounts for skin pigmentation of a user by optimizing the data captured by the optical sensor 103. Additionally, the skin pigmentation module may also account for other source of personal variance in light reflectance characteristics. In one instance, the user calibration module 406 may discount certain data artifacts or discrepancies based on the skin pigmentation of the user. In other instances, the user calibration module 406 may send a request to a microcontroller to increase or decrease the signal strength of a light emitter 302 housed in an optical sensor unit 103. Skin pigmentation of a user may be measured by a sensor 306 or can be input by the user on a computing device that is communicatively coupled to the processor 202.

The geometry offset module 408 optimizes data captured by the optical sensor by accounting for geometry and spacing of the light emitters 302 and sensors 306 housed in the device 100. Data captured by a sensor 306 varies based on the number and geometry of the light emitter 302 passing light within body tissues of a user. As such, the geometry offset module 408 optimizes the data captured by the optical sensor to account for the number, mode and geometry of the light emitters 302 and sensors 306.

The noise offset module 410 processes signals received from one or more sensor to identify signal noise identified at the one or more sensors. For example, if an acute motion is detected by the motion sensor 105 at a particular time, a peak detected by the optical sensor 103 at the same time may be discounted as being attributable to the motion of a user. In another embodiment, the noise offset module 410 can anticipate a peak in an optical signal based on a heart rate of the user. For example, if heart rate of a user is sixty beats a minute, the noise offset module 410 may calculate that the next beat to be detected by the optical sensor 103 will occur during a time window that corresponds to a heart rate of 40 to 80 beats per minute. In such an instance, the noise offset module 410 can dynamically adjust the optical sensor 103 to identify peaks found in a set of samples corresponding to a particular heart rate range and thereby identifying peaks occurring outside that interval as signal noise.

The feedback module 412 generates optimized data to display to a user. In one embodiment, the feedback module 412 receives optimized biometric data, including blood flow, blood flow frequency, user motion data, skin conductivity data, skin and ambient temperature data and provides the data to a user in one or more formats. For example, the feedback module 412 may convert the blood flow velocity or flow frequency data to heart rate data to present to a user. Similarly, the feedback module 412 may convert the skin conductivity data to an indication of stress level and motion data as activity level indication to display to a user. In one instance, the feedback module 412 converts and provides the data to substantially real-time as the data captured by the one or more sensors for internal signal calibration, optimization, for direct or indirect feedback to the wearer, storage or transmission. As described in the specification, it is an advantage of the device to capture and display substantially real-time data to a user on a single device 100. The captured data may be used to provide feedback on goals of a user, progress, alerts on events, alerts to connect to a web server to additional information, audio/visual or other feedback and to communicate with a user.

### Method of Calculating Biometric Data

FIG. 5 illustrates a method of calculating biometric data of a user based on signals received from one or more sensors housed in a device 100. In one embodiment, the process receives 502 input signals from a GSR sensor 102. The input signal may include information about sweat levels of a user as measured by the GSR sensor. The processor 202 may identify a state associated with physical activity of a user, emotional arousal or other conductivity changing events.

The process also receives 504 input signals from an ambient temperature sensor 104 and input signals 505 from a skin temperature sensor 106. The input signals may include information about skin temperature of a user as measured locally by the skin temperature sensor 106 and ambient temperature around the user. The skin temperature of a user and ambient temperature may be used to identify contextual data about a user, such as activity levels of the user, etc.

The process receives 506 input signals from a motion sensor 105 housed in a device 100. The motion signal may include information about a rectilinear and rotational acceleration, motion or position as well as rectilinear and rotational speed or vector of a user. Additionally, the process receives 508 input signals from an optical sensor 103. The input signal may include information associated with a pulse measure by the optical sensor 103 at a location on the body of a user.

In one embodiment, the process calculates 510 biometric data associated with a user based on information received from the one or more sensors. For example, the process calculates 510 a pulse rate of a user based on signals received from the optical sensor 103. Additionally, the process may discount signals received from the sensors that are likely signal noise. For example, if the process determines that a heart rate of a user is in a particular range, it may identify signal peaks within a corresponding interval and discount signal peaks outside of the corresponding interval range. Similarly, if the process identifies an acute movement at a particular time based on a signal received from the motion sensor, the process may discount an optical signal peak at the same time and attribute it to the motion of a user. Additionally, the process calculates 510 a biometric data of a user by including an offset for skin pigmentation of a user which may affect the reflected light received by the optical sensor 103. Additionally, the process calculates 510 biometric data of a user by accounting for other sources of personal variance in light reflectance characteristics. The biometric data calculated 510 by the process may include one or more of heart rate, skin temperature, ambient temperature, heart rate variability measure, blood flow rate, pulse oximetry, caloric burn rate or count, activity level, step count, stress level, blood glucose level and blood pressure.

The process sends 512 the calculated biometric data to a display. The display may be housed on the device 100 or may be located remotely from the device 100. The biometric data may be sent to the display using a wired or a wireless connection as described in reference to FIG. 2, such that the display may provide a heart rate, skin temperature, ambient temperature, heart rate variability measure, blood flow rate, pulse oximetry, caloric burn rate or count, activity level, step count, stress level, blood glucose level and blood pressure of a user in a display interface.

### Additional Configuration Considerations

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Certain embodiments are described herein as including logic or a number of components, modules, or mechanisms, for example, as described in FIGS. 3 and 4. Modules may constitute either software modules (e.g., code embodied on a machine-readable medium or in a transmission signal) or hardware modules. A hardware module is tangible unit capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations. Hence, by way of example, the modules described in FIGS. 3 and 4 can be structured electronically in one or more ASICs.

Further, the term “hardware module” should be understood to encompass a tangible entity, be that an entity that is physically constructed, permanently configured (e.g., hardwired), or temporarily configured (e.g., programmed) to operate in a certain manner or to perform certain operations described herein. As used herein, “hardware-implemented module” refers to a hardware module. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods described herein may be at least partially processor-implemented. For example, at least some of the operations of a method may be performed by one or processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The one or more processors may also operate to support performance of the relevant operations in a “cloud computing” environment or as a “software as a service” (SaaS). For example, at least some of the operations may be performed by a group of computers (as examples of machines including processors), these operations being accessible via a network (e.g., the Internet) and via one or more appropriate interfaces (e.g., application program interfaces (APIs).)

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Some portions of this specification are presented in terms of algorithms or symbolic representations of operations on data stored as bits or binary digital signals within a machine memory (e.g., a computer memory). These algorithms or symbolic representations are examples of techniques used by those of ordinary skill in the data processing arts to convey the substance of their work to others skilled in the art. As used herein, an “algorithm” is a self-consistent sequence of operations or similar processing leading to a desired result. In this context, algorithms and operations involve physical manipulation of physical quantities. Typically, but not necessarily, such quantities may take the form of electrical, magnetic, or optical signals capable of being stored, accessed, transferred, combined, compared, or otherwise manipulated by a machine. It is convenient at times, principally for reasons of common usage, to refer to such signals using words such as “data,” “content,” “bits,” “values,” “elements,” “symbols,” “characters,” “terms,” “numbers,” “numerals,” or the like. These words, however, are merely convenient labels and are to be associated with appropriate physical quantities.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

Upon reading this disclosure, those of skill in the art will appreciate still additional alternative structural and functional designs for a system and a process for optimizing biometric data captured by one or more sensors housed in a device by accounting for actions or items that may distort the data, through the disclosed principles herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

What is claimed is:

1. An apparatus for measuring biometric data of a user on a device secured to a user, the apparatus comprising:
a galvanic skin response sensor measuring a state of the user associated with physical activity, emotional arousal or other conductivity changing event;
an ambient temperature sensor measuring ambient temperature associated with surroundings of the user, the ambient temperature providing contextual data about biometric measurements of the user;
a skin temperature sensor measuring temperature of the user, the measured temperature used to measure the biometric data of the user;
a motion sensor measuring motion of the user, the motion sensor including a multi-axis accelerometer measuring a magnitude and direction of acceleration of the motion;
a light emitter transmitting light to body of the user, the emitted light of a particular wavelength, geometry, intensity and mode;
an optical sensor receiving light passed by the wavelength selection filter and converting the received light to data; and
a processor receiving data from the galvanic skin response sensor, the ambient temperature sensor, the skin temperature sensor, the motion sensor and the optical sensor and computing the biometric data of the user, the biometric data including an offset for motion experienced by at least one sensor, an offset for skin pigmentation of the user, the skin pigmentation effecting light received by the optical sensor and accounting for other sources of personal variance in light reflectance characteristics, an offset for geometry of the light emitter.

2. The apparatus of claim 1, further comprising a wavelength selection filter selectively passing light received from the user tissues and body fluids, the light selected based on its wavelength.

3. The apparatus of claim 1, further comprising:
a display presenting biometric data calculated by the processor, the biometric data including one or more of heart rate, skin temperature, heart rate variability measure, blood flow rate, pulse oximetry, stress level or stress events, emotional arousal levels or events, blood glucose level and blood pressure.

4. The apparatus of claim 1, further comprising:
a power supply enabled to provide power to the apparatus, the power supply capable of harvesting energy from at least one of a variety of sources.

5. An apparatus for measuring biometric data of a user on a device secured to a user, the apparatus comprising:
a light emitter transmitting light to body of the user, the emitted light of a particular wavelength, geometry, intensity and mode for reflection from a body of the user;
an optical sensor adapted to receive the light transmitted into the body of the user by the emitter and received back from the body of the user, the received light varying in intensity based on a flow of blood within the body of the user, the optical sensor further adapted to convert the received light to a voltage that corresponds to the intensity of the light;
a processor adapted to receive data from the optical sensor and compute biometric data about the user based data representing the light received from the body of the user, the data being varied based on the bioprocesses of the user and offset the received data based on characteristics of the user body affecting the received light.

6. The apparatus of claim 5, further comprising:
a galvanic skin response sensor adapted to measure a state of the user associated with at least one of physical activity and emotional arousal; and
a processor adapted to receive data from the galvanic skin response sensor and computing a measurement associated with skin conductivity of the user, the measurement providing an indication of arousal or other skin conductivity changing events of the user.

7. The apparatus of claim 5, further comprising:
an ambient temperature sensor adapted to measure ambient temperature associated with surroundings of the user, the ambient temperature providing contextual data about biometric measurements of the user; and
a processor adapted to receive data from the ambient temperature sensor and computing a measurement associated with ambient temperature near the user, the processor discounting the temperature of the user.

8. The apparatus of claim 5, further comprising:
a skin temperature sensor adapted to measure temperature of the user, the biometric data of the user comprising the temperature of the user; and
a processor adapted to receive data from the skin temperature sensor and computing temperature of the user based on the received data.

9. The apparatus of claim 5, further comprising:
a motion sensor adapted to measure motion of the user, the motion sensor including a multi-axis accelerometer measuring a magnitude and direction of acceleration of the motion; and
a processor adapted to receive data from the motion sensor and computing a measurement of movement of the user, the movement measured in at least one direction based on a number and type of motion sensors.

10. The apparatus of claim 9, wherein the processor is configured to measure at least one of rectilinear and rotational acceleration, motion, position and a change in rectilinear and rotational speed of the user.

11. The apparatus of claim 5, further comprising:
a wavelength selection filter adapted to selectively pass light received from the user tissues and body fluids, the light selected based on its wavelength; and
a processor adapted to receive data from the wavelength selection filter and computing the biometric data of the user, the biometric data including a measurement associated with the light received by the optical sensor.

12. The apparatus of claim 5, further comprising:
at least two light emitters, each light emitter adapted to emit light of differing wavelength, geometry, intensity and mode;
at least two optical sensors, each optical sensor associated with a corresponding light emitter and adapted to receive light of a particular wavelength, geometry, intensity and mode emitted by a corresponding light emitter.

13. The apparatus of claim 5, wherein biometric data includes a heart rate of the user.

14. The apparatus of claim 5, wherein the processor further configured to provide for display of the biometric data.

15. A computer-readable method for measuring biometric data of a user on a device secured to a user, the method comprising:
a light emitter transmitting light to body of the user, the emitted light of a particular wavelength, geometry, intensity and mode for reflection from a body of the user;
an optical sensor adapted to receive the light transmitted by the emitter and received from the body of the user, the received light varying in intensity based on a flow of blood within the body of the user, the optical sensor further adapted to convert the received light to a voltage that corresponds to the intensity of the light;
a processor adapted to receive data from the optical sensor and compute biometric data about the user based data representing the light received from the body of the user, the data being varied based on the bioprocesses of the user and offset the received data based on characteristics of the user body affecting the received light.

16. The computer-readable method of claim 15, further comprising:
measuring a state of the user associated with at least one of a physical activity and emotional arousal;
receiving data from an galvanic skin response sensor; and
computing a measurement associated with skin conductivity of the user, the measurement providing an indication of arousal or other conductivity changing events of the user.

17. The computer-readable method of claim 15, further comprising:
measuring ambient temperature associated with surroundings of the user, the ambient temperature providing contextual data about biometric measurements of the user;
receiving data from an ambient temperature sensor;
computing a measurement associated with ambient temperature near the user; and
discounting the temperature of the user.

18. The computer-readable method of claim 15, further comprising:
measuring temperature of the user, the biometric data of the user comprising the temperature of the user;
receiving data from an skin temperature sensor; and
computing a measurement associated with the temperature of the user.

19. The computer-readable method of claim 15, further comprising:
measuring motion of the user using motion sensor including a multi-axis accelerometer for measuring a magnitude and direction of acceleration of the motion;
receiving data from the motion sensor; and
computing a measurement of movement of the user, the movement measured in at least one direction based on a number and type of motion sensors.

20. The computer-readable method of claim 19, further comprising measuring at least one of rectilinear and rotational acceleration, motion, position and a change in rectilinear and rotational speed of the user.
